(19) United States
(12) Patent Application Publication
(10) Pub. No.: US 2004/0109855 A1
(43) Pub. Date: Jun. 10, 2004

(54) Therapeutic antibodies with reduced side effect

(76) Inventors: Herman Waldmann, Oxford (GB); Mark Raymond Frewin, Oxford (GB); Lisa Kim Gilliland, Merced, CA (US); Luis Ricardo Simoes Da Silva Graca, Oxford (GB)

Correspondence Address: CARELLA, BYRNE, BAIN, GILFILLAN, CECCHI,, STEWART & OLSTEIN, 6 Becker Farm Road, Roseland, NJ, 07068, US

(21) Appl. No.: 10/615,718

(22) Filed: Jul. 9, 2003

Related U.S. Application Data
(60) Provisional application No. 60/397,934, filed on Jul. 23, 2002.

(51) Int. Cl. A61K 39/395; A61K 38/17

(52) U.S. Cl. 424/130.1; 514/12

(57) ABSTRACT

A therapeutic protein, such as a therapeutic antibody, that is modified with a compound that inhibits binding of the protein to its therapeutic target, thereby reducing side effects caused by the protein.

This application claims priority based on provisional application Serial No. 60/397,934, filed Jul. 23, 2002, the contents of which are incorporated by reference in their entirety.

### FIELD OF THE INVENTION

The present invention relates to therapeutic antibodies and to a method for reducing the side effects thereof; in particular, those that result from cytokine release.

### BACKGROUND

In protein therapy, such as antibody therapy, the use of the antibody results in unwanted side effects; for example, those that result from cytokine release. As a result, there is a need for therapeutic proteins with reduced side effects.

### STATEMENT OF THE INVENTION

In accordance with one aspect of the present invention, there is provided a modified therapeutic antibody wherein the modified therapeutic antibody as compared to the unmodified antibody has a limited reduced binding to its target antigen, which reduced binding reduces the side effects thereof such as those resulting from cytokine release. In a preferred embodiment, the reduced binding is such that over time the binding of the antibody to the target is increased.

According to one aspect, the present invention is directed to a therapeutic antibody which comprises a therapeutic antibody having a specific therapeutic effect wherein the antibody has been subject to a limited obstruction of its antibody-combining site which reduces the binding of the antibody for its natural target and wherein following administration to a host the antibody is capable of achieving the said therapeutic effect, whereby the reduction of the binding of the antibody for its natural target reduces the side effects of the antibody.

Thus, in accordance with an aspect of the invention, there is provided a pharmaceutical in the form of a therapeutic antibody wherein the therapeutic antibody includes an antibody combining site (ACS) for a therapeutic target and the antibody is modified with a compound that inhibits the binding of the therapeutic antibody to the therapeutic target in a limited manner.

In one such embodiment there is provided a therapeutic antibody that is modified to include a compound that is reversibly bound to the antibody combining site of the antibody, with the target antigen competing with the compound for binding to the ACS upon administration of the antibody, or the binding of the antigen otherwise being inhibited by the compound, whereby binding of the antibody to the target is inhibited. In this manner, the amount of the modified antibody that becomes bound to the target antigen in the initial period after administration is less than would have become bound if the antibody was administered in its non-modified form. As the compound is displaced from the ACS as a result of competitive binding, or the inhibitory affect of the compound is otherwise reduced, the amount of antibody that becomes bound to the target antigen increases. By inhibiting the binding of the antibody, with the amount of antibody that is bound to the target increasing over time, the modified antibody is capable of reducing and/or essentially eliminating side effects such as those that result from cytokine release and is also capable of accomplishing the desired therapeutic effect.

In one embodiment, the modified antibody has an avidity for the target that is less than the avidity for the target of the unmodified antibody. The avidity is reduced in an amount that is effective for reducing and/or eliminating side effects against the therapeutic antibody while producing the desired therapeutic effect by binding to the therapeutic target.

The term “therapeutic” as used herein encompasses both treating an existing disease condition or disorder and preventing and/or reducing the severity of a disease, condition or disorder.

A therapeutic target is the antigen to which the antibody binds, which antigen may or may not be present on a tissue or cells. The compound that is combined with the therapeutic antibody for inhibiting binding to the target may inhibit such binding by binding to the ACS and/or by binding or blocking access to the ACS; e.g., by steric hindrance.

The compound may be combined with the antibody by linking the compound to the antibody and/or by binding of the compound to the ACS. In one embodiment, the compound is linked or tethered to the antibody and also binds to the ACS. In another embodiment, the compound is linked to the antibody without binding to the ACS and inhibits binding of the antibody to the target by inhibiting access to the ACS; e.g., by steric hindrance. In one non-limiting embodiment, the compound is linked to only one of the chains of the antibody. The compound that is used to inhibit binding may be a linker compound which does not include a compound that binds to the ACS, provided that such linker initially inhibits binding of the antibody to the target antigen.

The therapeutic antibody may be used as a therapeutic in humans and may be a non-human antibody e.g. one raised in a rodent.

The compound functions to inhibit binding of the antibody to the target whereby immediately after administration there is a limited reduction of the amount of antibody that binds to the target as compared to the amount of antibody that would bind without the presence of the compound. The amount of antibody that becomes bound to the target increases over time whereby in effect there is a temporary blocking of the ACS that inhibits the amount of antibody that binds to the target.

The temporary blockade of the ACS (a blockade that initially reduces the amount of antibody that binds to the target, with such amount increasing with time) may be effected by the following, including;

(i) Temporary occupancy with molecules such as the defined antigen or a domain thereof, low affinity antigenic peptides or mimotopes by pre-incubation in-vitro, that might gradually dissociate in-vivo, such that the antibody would gradually accumulate on cell-bound or other “target” antigen if the association and dissociation constants were favourable by comparison with the “obstructive” element; or

(ii) Temporary occupancy with molecules such as the defined antigen or a domain thereof, low affinity antigenic peptides or mimotopes which may be attached by flexible linkers. Once administered in-vivo the antibody would gradually accumulate on cell-bound or other “target” antigen if the association and dissociation constants were favourable by comparison with the “obstructive” element; or

(iii) Chemical drugs which may bind non-covalently in the ACS and be able to dissociate in-vivo; or

(iv) Other changes that might temporarily obstruct the ACS; for example, temporarily blocking access to the ACS without the compound binding to the ACS.

Such a modification would interfere with antibody accumulation on the target antigen for a limited period, which would be enough to ensure that the administered therapeutic antibody has reduced side effects while allowing for the antibody to achieve the desired therapeutic effect, i.e., accumulate on the target antigen in an amount to produce such effect. Removal of the modification may also occur by the host's own physiological and biochemical processes such as pH changes, enzymatic cleavage within the host, natural competition with host antigens bound to cells. For example a peptide mimotope could be linked to the antibody H or L chain by a linker which carries an enzyme-degradable motif, susceptible to cleavage by host enzymes in-vivo, such as for example, leukocyte elastase.

According to one particularly advantageous embodiment of the invention the linker is cleaved by an enzyme which occurs only or preferentially at the desired site of action of the therapeutic antibody thereby providing selective delivery of the therapeutic antibody to the desired site of action. For example a linker cleaved by leukocyte elastase would be appropriate for an antibody whose intended site of action is inflamed joints. Alternatively, soluble antigen or mimotope might dissociate more easily at low pH within the site of a tumour which may also provide selective delivery of the antibody to the desired site of action. Alternatively, a low affinity mimotope attached by an inert linker may naturally dissociate in-vivo, and reassociation may be prevented by the ACS interacting with the natural antigen on host cells.

A linker may also be designed to contain a site cleavable by administration of a therapeutic enzyme. For example, a linker may be designed to contain a cleavage site for TPA or streptokinase. The rate of cleavage (and therefore, the rate of accumulation on cells) may be controlled by controlling the amount and rate of administration of TPA or streptokinase. Along the same lines, if the linker is cleaved by a naturally occurring enzyme in vivo, similar control may be achieved by administration of an enzyme inhibitor which would block cleavage.

Preferably, the native antigen, domains thereof, and peptide fragments or mimotopes are used to modify the ACS. The latter may be generated from peptide libraries either synthetically or biologically-derived. Non-covalently binding chemicals can be screened from natural or synthetic libraries or from other available products, for their ability to inhibit antibody binding to its antigen or a surrogate equivalent. The linkers which may be used are preferably flexible, but could be enzymatically cleavable and/or degradable by the body or over a set time period. [see comment above]

The present invention is also directed to antibodies as described above further comprising an Fc region designed to reduce interaction with the complement system and with specialised cell receptors for the Fc region of immunoglobulins (FcR receptors). This will be useful for many antibodies where cell lysis is not essential, such as blocking or agonist antibodies.

According to a further aspect, the invention provides an antibody as defined above for use in therapy.

According to a still further aspect, the invention provides the use of an antibody as defined above in the manufacture of a medicament for use in the treatment of a mammal to achieve the said therapeutic effect. The treatment comprises the administration of the medicament in a dose sufficient to achieve the desired therapeutic effect. The treatment may comprise the repeated administration of the antibody.

According to a still further aspect, the invention provides a method of treatment of a human comprising the administration of an antibody as defined above in a dose sufficient to achieve the desired therapeutic effect and reduce and/or eliminate side effects that result, for example, from immediate and massive accumulation of antibody on target cells; in particular, cytokine release. The therapeutic effect may be the alleviation or prevention of diseases which may include cancer, chronic inflammatory diseases such rheumatoid arthritis, autoimmune diseases such as diabetes, psoriasis, multiple sclerosis, systemic lupus and others, allergic diseases such as asthma, cardiovascular diseases such as myocardial infarction, stroke and infectious diseases. Indeed any disease where continuous or repeated doses of a therapeutic antibody are contemplated.

Temporary modification of the type described above may also be applicable to therapeutic proteins other than antibodies whose activity depends on a biologically active site which can be transiently blocked and where the activity of this site determines immunogenicity. Examples of such therapeutic proteins include hormones, enzymes, clotting factors, cytokines, chemokines, and immunoglobulin-based fusion proteins.

When covalently linking the compound to the antibody, in one embodiment, the compound is preferably linked to only one of the two arms of the antibody.

The term “antibody” as used herein includes all forms of antibodies such as recombinant antibodies, humanized antibodies, chimeric antibodies, single chain antibodies, monoclonal antibodies etc. The invention is also applicable to antibody fragments that are capable of binding to a therapeutic target.

In one embodiment, a compound (which may be a peptide or other molecule that is capable of binding to the ACS of the antibody) is reversibly bound to the antibody binding or combining site of the antibody that is to be administered to a person. The compound occupies the binding site of the antibody for the antigen and thereby inhibits binding of the antibody to the antigen. Since the compound is reversibly bound to the antibody binding site and is selected to have a limited reduction in antibody binding, the antibody is capable of binding to the antigen against which the antibody is directed.

In one embodiment, the compound that is selected for binding to the antibody combining site of the antibody is one whereby the antibody avidity for the compound is less than the antibody avidity for the antigen. In this manner, when the antibody is initially administered, there will be reduced binding of the antibody to the antigen, as compared to the binding that would occur in the absence of the compound, with such binding increasing over time.

Applicant has found that reduction of side effects (such as those resulting in cytokine release) to a therapeutic antibody can be accomplished by administering an antibody that is capable of effectively binding to the antigen producing the desired therapeutic effect, provided that during the period that immediately follows administration of the antibody, the amount of the antibody that binds to the antigen is reduced, with such amount being increased from the reduced amount over time.

Thus, unlike the prior art, in accordance with the invention, an antibody that is capable of performing its therapeutic function also reduces side effects, such as cytokine release caused by the antibody by initially reducing the amount of the therapeutic antibody binding to the antigen followed by an increase in the amount of the therapeutic antibody binding.

The compound that is used for binding to the antibody combining site in a manner that initially reduces the amount of antibody binding to the antigen may be a peptide. The peptide may be identical to or different from a corresponding peptide portion of the antigen to which the therapeutic antibody binds. The appropriate peptide for an antibody may be selected by testing a panel of peptides in an inhibition of binding assay with respect to the antibody and its antigen. These and other procedures should be known to those skilled in the art based on the teachings herein.

In one embodiment, the antibody combined with the compound has an avidity for the target antigen that is less than the avidity of the non-modified antibody for the target antigen. The relative avidity of the modified antibody and the unmodified antibody may be determined by an inhibition of binding assay using fifty percent binding inhibition as an end point. A modified antibody has a reduced avidity if there is an increase in the amount of modified antibody as compared to the amount of unmodified antibody required to produce a fifty percent inhibition of the binding of a labeled unmodified antibody to the target antigen.

The avidity of the modified antibody is reduced in an amount that is effective for reducing and/or essentially eliminating side effects, in particular those caused by cytokine release and the modified antibody has an avidity for the target that is effective for producing the desired therapeutic effect. In general, the avidity is reduced by at least 4-fold and in general by no more than 100-fold. It is to be understood that there may be a greater reduction in avidity. Thus, in accordance with an aspect of the invention, the antibody is modified to reduce side effects and, in the case where it is desired to only reduce side effects, the reduction in binding is limited to an amount to achieve such result; i.e., side effect reduction can be achieved without reducing or eliminating an antibody response against the modified antibody.

In one non-limiting embodiment, the compound may inhibit binding of the modified antibody by providing a modified antibody with a reduced affinity for the target antigen as compared to the unmodified antibody. In one non-limiting embodiment, the modified antibody may have an affinity for the antigen to which it is to be bound that is at least two or at least five-fold less than the affinity of the unmodified antibody. In accordance with an embodiment of the invention, side effects can be reduced by reducing the affinity in a limited amount, for example, by no more than 100 fold and without eliminating an antibody response against the modified antibody. It is to be understood, however, that the affinity may be reduced in greater amounts, although, however, such greater reduction is not required in order to reduce side effects caused by cytokine release.

The modified antibody is employed in an amount that is effective for both producing the desired therapeutic effect and for reducing side effects. In general, without limiting the present invention, the modified antibody is administered in an amount such that the quantity of the antibody administered during the 24-hour period that begins when the antibody is first administered is at least 50 mg and in general at least 100 mg and more generally at least 200 mg.

The therapeutic antibody may be employed in combination with a pharmaceutically acceptable carrier. The use of a suitable carrier is deemed to be within the skill of the art from the teachings herein.

The present invention is also directed to a therapeutic protein which comprises a protein having a specific therapeutic effect wherein the protein has a biologically active site which has been subject to a temporary obstruction which reduces the binding of the protein for its natural target and wherein following administration to a host the protein achieves the said therapeutic effect, whereby the reduction of the binding of the protein for its natural target reduces side effects.

The following Examples illustrate the invention.

The humanised anti-CD52 antibody, CAMPATH-1H, was used in the following experiments. Various constructs were made using the CAMPATH-1H antibody and the following methodology.

Generation of Reduced-Binding Variants of CAMPATH-1H:

The cloning of the V-regions of the humanised antibody CAMPATH-1H specific for the human CD52 antigen is performed as described in Gilliland et al. 1999 The Journal of Immunology 162:33663-3671. The methodology is based on that of Orlandi et al. 1989 PNAS 86:3833, using the polymerase chain reaction (PCR). The wild-type humanised CAMPATH-1 light chain was cloned into the vector pGEM 9 (Promega) and used as a PCR template for site-directed mutagenesis.

A flexible linker (Gly4Ser ×2) was added to the amino-terminal end of the light chain between the CAMPATH-1H leader sequence and CAMPATH-1H VL sequence using the oligonucleotide primers PUCSE2 and Link L-3′+Link-L-5′ and PUCSE REV. The resulting fragments were PCR assembled using primers PUCSE2+PUCSE REV to give full length Linker-CP-1H light chain which could be cloned into expression vector as Hind111/Hind 111 fragment.

The Linker-CP-1H light chain construct was then used as a PCR template to generate the CD52 Mimotope (QTSSPSAD) and the CD52 Mimotope Mutant 9 (QTSAAAVD) constructs. Primers PUCSE2 and CD52MIM-3′+CD52MIM-5′ and PUCSE REV were used to give Mimotope-CP-1H light chain construct. Primers PUCSE2 and MIMMut9-3′+MIMMut9-5′ and PUCSE REV were used to give Mimotope Mutant 9-CP-1H light chain construct.

Linker-Only-CP-1H, Mimotope-CP-1H, and MimMut9-CP-1H mutants were transferred to pBAN-2, a derivative of the pNH316 mammalian expression vector containing neomycin selection (Page et al. 1991 Biotechnology 9:64-68) and PEE 12 a mammalian expression vector containing the Glutamine Synthetase gene for selection (Bebbington et al. 1992 Biotechnology 10:169-175).

Subconfluent dhfr⁻ Chinese Hamster Ovary cells (Page et al. 1991 Biotechnology 9:64-68) or NSO mouse myeloma cells (ECACC cat no 8511503, Meth Enzymol 1981, 73B,3) were co-transfected with the light chain mutants and the CAMPATH-1H heavy chain construct human IgG1 constant region.

CAMPATH-1H heavy chain constructs were expressed in pRDN-1, a variant of the pLD9 mammalian expression vector with a dhfr selectable marker (Page et al. 1991 Biotechnology 9:64-68), and PEE 12. Transfection was carried out using LipofectAMINE PLUS reagent (Life Technologies) following the manufacturers recommendations.

Human IgG1 constant region was derived from the wild type Glm (1,17) gene described by Takahashi et al. 1982 Cell 29: 671-679.

Heavy and Light chain transfectants were selected in hypoxanthine-free IMDM containing 1 mg/ml G418+5% (v/v) dialysed fetal calf serum. Resulting selected cells were screened for antibody production by ELISA and for antigen binding to human T cell clone HUT 78 (Gootenberg J E et al. 1981 J. Exp. Med. 154:1403-1418) and CD52 transgenic mice.

Cells producing antibody were cloned by limiting dilution, and then expanded into roller bottle cultures. The immunoglobulin from approximately 15 litres of tissue culture supernatant from each cell line was purified on protein A, dialysed against PBS and quantified.

List of Primers Used

| | |
|---|---|
| PUCSE-2 | 5′-CAG AGA TGG GTA AGG AGA AAA TAG-3′ |
| PUCSE REV | 5′-GGA GTG AGC GGA AGG GAA T-3′ |
| LINK-L3′ | 5′-GGT TCG GGC TGC ACC GGA TCC GCC ACC |
| | TCC TTG GGA GTG GAC AGC TGT AGC TGT |
| | TGC TAG-3′ |
| LINK-L5′ | 5′-GGA GGT GGG GGA TGC GGT GGA GGG GGA |
| | AGC GAG ATC GAG ATG ACC GAG AGC GGA AGT |
| GD52MLM-3′ | 5′-GTG TGG TGA TGG GGT GCT GGT TTG GGA |
| | GTG GAG AGC TGT AGC TGT TGG-3′ |
| GD52Mim-5′ | 5′-GAA AGG AGC AGG GGA TGA GGA GAG GGA |
| | GGT GGC GGA TGG GGT GGA GGA-3′ |
| MimMut9-3′ | 5′-GTC TAG TGG TGG GGG GGT GGT TTG GGA |
| | GTG GAG AGG TGT AGG TGT TGG-3′ |
| MimMut9-5′ | 5′-CAA ACC AGO GOG GCA GGA GTA GAG GGA |
| | GGT GGC GGA TOG GGT GGA GGA-3′ |

Constructs and Cell Lines Produced

TF CHO/CP-1H IgG1/MIM and TF NSO/CP-1H IgG1/MIM (MIM IgG1)

CD52 Mimotope QTSSPSAD tethered to CAMPATH-1H light chain V-region by flexible Glycine4 Serine ×2 Linker+Campath-1H heavy chain with wild type human IgG1 constant region. Cloned into expression vector PEE12 (Celltech) for NSO-produced antibody and pRDN-1 and pBAN-2 expression vectors Wellcome for CHO-produced antibody.

TF CHO/CP-1H IgG1/Link (Linker)

Flexible Glycine4 Serine ×2 Linker on CAMPATH-1H light chain V-region+CAMPATH-1H heavy chain with wild type human IgG1 constant region. Cloned into pRDN-1 and pBAN-2 expression vectors (Wellcome) for CHO-produced antibody.

TF CHO/CP-1H IgG1/MIM-MUTANT 9 (MIM-MUTANT 9-IgG1)

CD52 Mimotope Mutant 9 (QTSAAAVD) tethered to CAMPATH-1H light chain V-region by flexible Glycine4 Serine ×2 Linker+CAMPATH-1H heavy chain with wild type human IgG1 constant region. Cloned into expression vectors pRDN-1 and pBAN (Wellcome) for CHO-produced antibody.

TF CHO/CO-1H IgG1 (CAMPATH-1H)

Wild type CAMPATH-1H light chain V-region+CAMPATH-1H heavy chain with wild type human IgG1 constant region. Cloned into expression vectors pRDN-1 and pBAN-2 (Wellcome) for CHO-produced antibody.

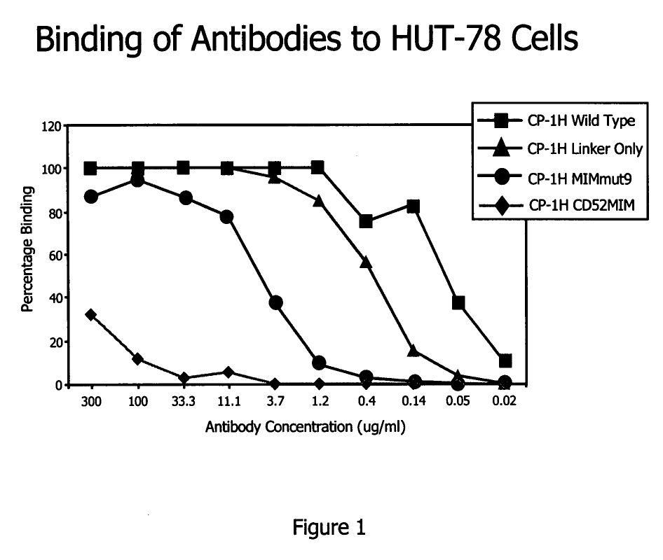

FIG. 1 shows the binding abilities of the various antibody constructs to CD52-bearing HUT cells. CP-1H Wild Type has a binding efficiency approximately 5 times greater than binding of CP-1H Linker alone, approximately 100 times greater than CP-MIMmut9, and approximately 10,000 times greater than CP-CD52MIM.

Effector Function Assessment

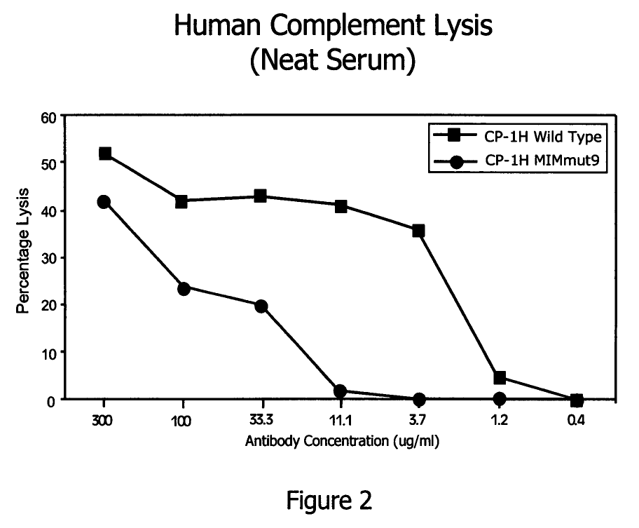

CP-1 H MIMmut9 effector function was assessed by testing its ability to kill the CD52 bearing T cell line, HUT78, by complement lysis. CP-1H wild type was used as a positive control. 100 ul of HUT78 cells at 10⁶ cells/ml were mixed with various concentrations of each antibody ranging from 0.4 ug/ml to 300 ug/ml. This was followed by the addition of 100 ul of fresh, undiluted (neat) human serum as a source of complement. After incubation at 37° C. for 45 minutes, propidium iodide was added to the mixture and the cells were analyzed by flow cytometry. The percentage of PI stained (lysed) cells was determined for each condition. The results shown in FIG. 2 demonstrate that CP-1H MIMmut9 retains the ability to mediate antibody effector function.

The modified and unmodified antibodies were tested to determine induction of cytokines as follows:

Cytokine Measurement

The purified antibodies were passed over a Detoxi-Gel column (Pierce) to remove endotoxin. All batches of antibody were then tested for endotoxin using the QCL 1000 LAL assay (Bio-Whittaker).

Ex-Vivo Whole Blood Assay

Blood from healthy laboratory workers was freshly drawn into Heparin. Whole blood samples were incubated with 100, 10 or 1 μg/ml of therapeutic antibody for five hours at 37° C. with vigorous shaking. The plasma and cells were then separated by centrifugation.

Plasma TNF-α levels were determined by human TNF-α immunoassay (R&D Systems), plasma IFN-γ levels were determined by human IFN-γ Elisa (BD Bioscience) and plasma IL-6 levels were determined by human IL-6 Elisa (BD Bioscience).

Tables 1 and 2 below report the results of such testing with the modified antibodies reducing release of cytokines.

TABLE I
TNF-α Levels (pg/ml) in Whole Blood
after 5 Hours at 37° C.

| | Amount of Antibody | | |
|---|---|---|---|
| Antibody | 100 μg/ml | 10 μg/ml | 1 μg/ml |
| CP-1H Wild Type(NSO) | 240 | 245 | >500 |
| CP-1H Wild Type(CHO) | 125 | 250 | >500 |
| CP-1H Linker Only (CHO) | 68 | 145 | 85 |
| CP-1H CD52MIM(NSO) | 0 | 0 | 0 |
| CP-1H CD52MIM(CHO) | 30 | 0 | 0 |
| CP-1H MIMmut9(NSO) | 60 | 28 | 0 |

TABLE II
Cytokine Levels in Whole Blood Assay after 5 hr
incubation at 37° C. with antibody

| Antibody | Cytokin (pg/ml) | | |
|---|---|---|---|
| (10 μg/ml) | TNF-α | IFN-γ | IL-6 |
| CP-1H Wild Type(NSO) | 245 | 262 | NT |
| CP-1H Wild Type(CHO) | 250 | 275 | 31 |
| CP-1H CD52MIM(NSO) | 0 | 0 | 0 |
| CP-1H MIMmut9(NSO) | 28 | 11 | 10 |

Numerous modifications and variations of the embodiments described herein are possible based on the teachings herein; therefore, the scope of the invention is not limited to such embodiments.

1. A pharmaceutical comprising:
(a) a therapeutic protein that binds to a therapeutic target, said protein being modified with a compound that inhibits binding of the protein to the therapeutic target, said modified protein being effective for reducing side effects caused by the protein and for producing a therapeutic effect by binding to the therapeutic target; and (b) a pharmaceutical carrier.

2. The pharmaceutical of claim 1 wherein the therapeutic protein is an antibody that includes an antibody combining site that binds to the therapeutic target.

3. The pharmaceutical of claim 2 wherein the compound is bound to the antibody combining site of the antibody.

4. The pharmaceutical of claim 3 wherein the compound is also linked to the antibody.

5. The pharmaceutical of claim 3 wherein the compound is a peptide.

6. The pharmaceutical of claim 5 wherein the avidity of the modified antibody combined with the peptide is at least 4-fold less than the avidity of the unmodified antibody and no more than 100-fold less.

7. The pharmaceutical of claim 6 wherein the antibody is an aglycosylated antibody.

8. The pharmaceutical of claim 7 wherein only one of the chains of the antibody has a peptide linked thereto that binds to the antibody combining site.

9. The pharmaceutical of claim 3 wherein the compound is reversibly bound to the antibody combining site, whereby the amount of antibody that binds to the target increases as the compound is displaced from the antibody combining site.

10. The pharmaceutical of claim 9 wherein the compound bound to the antibody combining site is also linked to the antibody.

11. The pharmaceutical of claim 10 wherein the compound is a peptide.

12. The pharmaceutical of claim 11 wherein the Fc portion of the antibody is aglycosylated.

13. The pharmaceutical of claim 11 wherein binding of the antibody to the Fc receptor is essentially eliminated.

14. The pharmaceutical of claim 10 wherein the antibody is a non-human antibody.

15. The pharmaceutical of claim 10 wherein the antibody is a chimeric antibody.

16. A process for treating a mammal, comprising administering to a mammal the pharmaceutical of claim 1 in an amount effective to both treat the mammal and reduce side effects against the protein.


SEQUENCE LISTING (8 sequences)

SEQ ID NO: 1 - DNA, 24 bp, Artificial
  PCR primer
cacagatgcgtaaggagaaaatac

SEQ ID NO: 2 - DNA, 19 bp, Artificial
  PCR primer
gcagtgagcgcaacgcaat

SEQ ID NO: 3 - DNA, 60 bp, Artificial
  PCR primer
gcttccgcctccaccggatccgccacctccttgggagtggacacctgtagctgttgctac

SEQ ID NO: 4 - DNA, 56 bp, Artificial
  PCR primer
ggaggtggcggatccggtggaggcggaagcgacatccagatgacccagagcccaag

SEQ ID NO: 5 - DNA, 48 bp, Artificial
  PCR primer
gtctgctgatgggctgctggtttgggagtggacacctgtagctgttgc

SEQ ID NO: 6 - DNA, 48 bp, Artificial
  PCR primer
caaaccagcagcccatcagcagacggaggtggcggatccggtggagga

SEQ ID NO: 7 - DNA, 48 bp, Artificial
  PCR primer
gtctactgctgcggcgctggtttgggagtggacacctgtagctgttgc

SEQ ID NO: 8 - DNA, 48 bp, Artificial
  PCR primer
caaaccagcgccgcagcagtagacggaggtggcggatccggtggagga